(19) United States
(12) Patent Application Publication
(10) Pub. No.: US 2008/0319371 A1
(43) Pub. Date: Dec. 25, 2008

(54) METHOD AND SYSTEM FOR TREATING OF ONYCHOMYCOSIS WITH AN APPLICATOR HAVING A GEL MEDICAMENT LAYER

(75) Inventors: Robert W. Etheredge, Natick, MA (US); Dennis I. Goldberg, Sudbury, MA (US); Shawn P. Davis, Boston, MA (US); Bireswar Chakraborty, Andover, MA (US); Michael S. Barsness, Oxford, MA (US)

Correspondence Address: NIXON & VANDERHYE, PC, 901 NORTH GLEBE ROAD, 11TH FLOOR, ARLINGTON, VA, 22203, US

(73) Assignee: TRANSPORT PHARMACEUTICALS, INC., Framingham, MA (US)

(21) Appl. No.: 12/142,234

(22) Filed: Jun. 19, 2008

Related U.S. Application Data
(60) Provisional application No. 61/040,366, filed on Mar. 28, 2008.
(60) Provisional application No. 60/952,676, filed on Jul. 30, 2007.
(60) Provisional application No. 60/944,907, filed on Jun. 19, 2007.

(51) Int. Cl. A61N 1/30; A61F 13/10

(52) U.S. Cl. 604/20; 427/2.31

(57) ABSTRACT

A method to electrokinetically deliver a medicament to a nail of a mammalian user including: forming a cast of the nail of the user wherein the cast is a reverse impression of the nail; applying a medicament matrix to the nail; sandwiching the medicament matrix between the cast and the nail; pressing the medicament matrix to the nail using the cast; applying electrical current to an electrical current path extending through the medicament matrix and the nail, and delivering medicament from the matrix into the nail by electrokinetic transporting the medicament along the current path.

### RELATED APPLICATIONS

This application claims the benefit of U.S. Provisional Patent Applications 60/944,907 filed Jun. 19, 2007; 60/952,676 filed Jul. 30, 2007, and 61/040,366 filed Mar. 28, 2008. The entirety of these applications are each incorporated by reference herein.

### BACKGROUND OF INVENTION

The present invention relates generally to applicators for electrokinetic mass transfer of substances to live tissue and particularly relates to an apparatus for electrokinetically delivering substances, e.g., a medicament, to a treatment site on or under a toenail or fingernail or area of hard skin.

Electrokinetic delivery of medicaments applies medication topically to the skin or nail, e.g., toenail, to reach a treatment site. One type of electrokinetic delivery mechanism is iontophoresis, which is the application of an electric field to the skin or nail to enhance the permeability of the skin or nail and thereby deliver ionic agents, e.g., ions of salts and other drugs, to the treatment site below the surface of the skin or nail. Iontophoretic, transdermal or transmucosal cutaneous delivery techniques can obviate the need for hypodermic injection of many medicaments. These delivery techniques avoid the concomitant problems associated with hypodermic injection, such as trauma, pain and risk of infection to the individual. Other types of electrokinetic delivery mechanisms include electroosmosis, electroporation, and electromigration, any or all of which are more generally known as electrotransport, electromolecular transport or iontophoretic methods. These techniques are collectively referred to herein as electrokinetic delivery methods.

Electrokinetic delivery methods may be problematic when applied to, for example, large areas of skin, such as on the face, or high impedance areas, such as toenails. Large skin treatment areas may be associated with skin conditions such as eczema, psoriasis and acne. To deliver medicament electrokinetically to a large skin treatment site, a relatively large medicament matrix is applied to the skin. A large electrical current is generally needed to electrokinetically drive medicament from the large area matrix into the skin. A large current is also typically required to overcome the high impedance of nails that is encountered in the electrokinetic delivery of onychomycosis medicament to a toenail.

Applying a large electrical current presents a risk of electrically irritating, sensitizing, or burning the skin, especially if the current is concentrated on a small skin area. A large area medicament matrix may be intended to contact a large area of skin. If the large area matrix is in contact with only a small area of skin, the large current applied to the matrix may become concentrated on and potentially burn the small skin area. In another example, the impedance, e.g. resistance, of a toenail is high and requires a high current to deliver onychomycosis medicament to treatment sites in or under the nail. Because of the high impedance of the toenail, a relatively high current is applied to a medicament matrix applied to the nail to deliver medicament to the nail. The medicament matrix applied to the toenail may also touch soft skin tissue next to the nail, which has a relatively low impedance as compared to the toenail. There is a risk that the high current will concentrate on the small area of soft skin that is in contact with the medicament matrix. The high current concentration may burn the skin adjacent the nail and in contact with the medicament matrix unless the current to those regions is controlled.

There is a long felt need for an electrokinetic device and method that delivers medicament to a large skin surface area and minimizes the potential for current concentration on the skin in contact with the medicament matrix. In particular, there is a need for an electrokinetic device capable of delivering medicament to a large skin contact area, to a toenail or fingernail, or to a curved area of the skin without applying a high concentration of current to any portion of the skin.

### SUMMARY OF INVENTION

A method is disclosed to electrokinetically deliver a medicament to a nail, e.g., a toenail, and/or surrounding skin (collectively referred to as the “toenail area” or “nail area”) of a mammalian user including: forming a cast of the nail area of the user wherein the cast is a reverse impression of the nail area; applying a medicament matrix to the nail area; sandwiching the medicament matrix between the cast and the nail area; pressing the medicament matrix to the nail area using the cast; applying electrical current to an electrical current path extending through the medicament matrix and the nail, and delivering medicament from the matrix into the nail and/or surrounding skin by electrokinetically transporting the medicament along the current path. The method may include extending an electrode through the cast and to the medicament matrix and applying a second electrode applied to an underside of the toe, wherein the current path includes the electrodes, the toenail and the toe.

The disclosed method may include forming the cast in an assembly having an upper housing with a well and a lower housing with an opposing well, wherein cast is formed by applying casting material to at least one of the wells and closing the upper housing over the lower housing while the nail and toe are sandwiched between the wells. After casting, the medicament matrix may be trimmed to conform to the nail area. Optionally, a separate non-conductive layer may be applied to the soft skin tissue adjacent the nail prior to the application of the electrical current to localize current delivery to the nail.

The medicament matrix is comprised of a porous material such as an open-cell foam, nonwoven or woven pad, or other absorptive material used to hold the drug formulation. Alternatively, the drug formulation may be used without an absorptive matrix if the formulation can be locally applied to the nail area without leakage from the site of placement. Examples of such formulation are described below and may include classes of phase change polymer solutions that flow at one temperature, a specific pH level, or a level of another condition and reversibly gel at another temperature, level pH, or different level of the another condition.

An electrode grid and medicament matrix assembly have been developed comprising: the electrode grid including a flexible array of electrodes; perforations through the grid and array of electrodes; a controller sensing current at each of the electrodes and capable of adjusting the current to each electrode, and the medicament matrix adjacent the electrode grid. The medicament matrix may be applied to a toenail area and sandwiched between the electrode grid and toenail area. The electrode grid is preferably a flexible sheet wherein the electrodes are arranged in a two-dimensional array with perforations interposed between the electrodes to provide three-dimensional flexibility.

An iontophoretic drug delivery system has been developed for the treatment of nail infections. The drug delivery system including: a housing which directs and localizes a composition having medicated formulation and hydrogel forming agents, such as a phase transforming polymer, to a nail area of a patient, wherein the formulation behaves as a solution at a temperature below about 50 degrees Celsius (C) (and most preferably below about 35 degrees C.) and behaves as a gel above about 50 degrees C. (and most preferably above about 35 degrees C.), and wherein the formulation is applied as a solution to the nail area and transformed to a gel after application to the nail area. The drug delivery system also includes integrated electrical connections and electronics for the application of therapeutic current to active electrode(s) applied to the gel, wherein current from the electrodes flows through the gel to deliver the medicament in the gel formulation to the nail or a treatment site below the nail.

### DESCRIPTION OF THE DRAWINGS

FIG. 1 is a perspective view of a casting made of toenails and electrokinetic medicament applicator pads formed using the casting.

FIGS. 2 to 8 are diagrams sequentially showing the operation of an embodiment of an electrokinetic medicament applicator for a toenail area.

FIG. 9 is a top view schematic diagram of a further embodiment of an electrokinetic medicament applicator pad having an electrode array.

FIG. 10 is a schematic cross-sectional diagram of a flexible electrode array and medicament pad applied to a toenail.

FIG. 11 is a schematic illustration of a front view of another embodiment of an electrokinetic medicament applicator applied to a toenail.

FIGS. 12 to 15 are front views of a toe with an electrokinetic medicament applicator and show a sequence of steps in which the compartment is being filled with RTV rubber (FIG. 12), a discharge of rubber indicating that that the compartment is filled (FIG. 13), loading a medicament matrix with medicament from a medicament delivery line connected to a source of medicament (FIG. 14), and the application of electrical current to the applicator to deliver medicament to the toe nail (FIG. 15).

FIG. 16 is a chart of a typical phase diagram of an E₉₉P₆₉E₉₉ block copolymer pluronic, referred to as F-127.

FIG. 17 is an exploded view of an applicator device and a toe to which the applicator device is to be applied.

FIG. 18 is a cross-sectional front view of the applicator device shown in FIG. 17 while applied to the toe and toenail.

FIG. 19 schematically shows an applicator pad in which a viscous gel formulation was cast directly on an electrode panel.

### DETAILED DESCRIPTION OF THE INVENTION

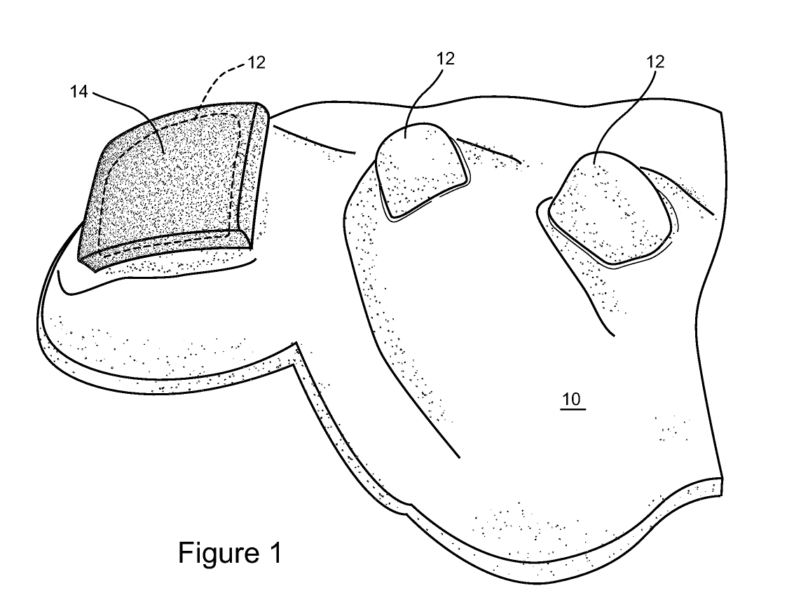

FIG. 1 is a diagram of an RTV rubber replica 10 taken of a three toenails of the foot of a human subject

The casting includes toenail reproductions 12 of the foot. The topography of the toenail is complex and varies among individuals. Further, infections may cause the toenail to deform. As the infection progresses, the toenail tends to thicken, roughen, and lose anchorage to the nail-bed of the underlying skin. In view of the varied topography of the toenail, it is difficult to fabricate a one-size fits all medicament matrix to apply to all toenails.

A medicament matrix intended to be a one-size fits all toenails will most likely be intentionally large relative to most toenails. A medicament matrix that is larger than the toenail will extend to the soft skin tissue of the toe that surrounds the toenail. A medicament matrix intended for a toenail that extends over the soft skin area potentially may apply excessive current to the skin. A medicament matrix that is smaller than a toenail will not cover the entirety of a toenail may not apply medicament to portion of the toenail not covered by the matrix.

Onychomycosis is the invasion of a toenail plate by a dermatophyte, yeast or nondermatophyte mould which are fungal infections of the nail. For effective onychomycosis treatment, it is desirable to have an antifungal agent, e.g., Terbinafine Hydrochloride or Fluconazole, penetrate the nail and saturate the subtending skin tissue.

Medicament to treat onychomycosis may be delivered electrokinetically to the toenail plate. In view of the high electrical impedance of the nail plate, a high voltage, such as over one hundred volts, may be required to electrokinetically deliver the medicament to subtending skin tissue for effective onychomycosis treatment.

The toenail to be given onychomycosis treatment often has a small surface area. There is a likelihood that the medicament matrix applied to the nail may touch the soft tissue adjacent the nail. Current concentration may occur where the matrix touches the soft tissue due to the high current applied by the active electrode and the relatively low resistance of the skin as compared to the resistance of the nail. Since current will seek the path of least resistance, current may traverse the medicament matrix to flow to the soft tissue to a much greater extent than the nail.

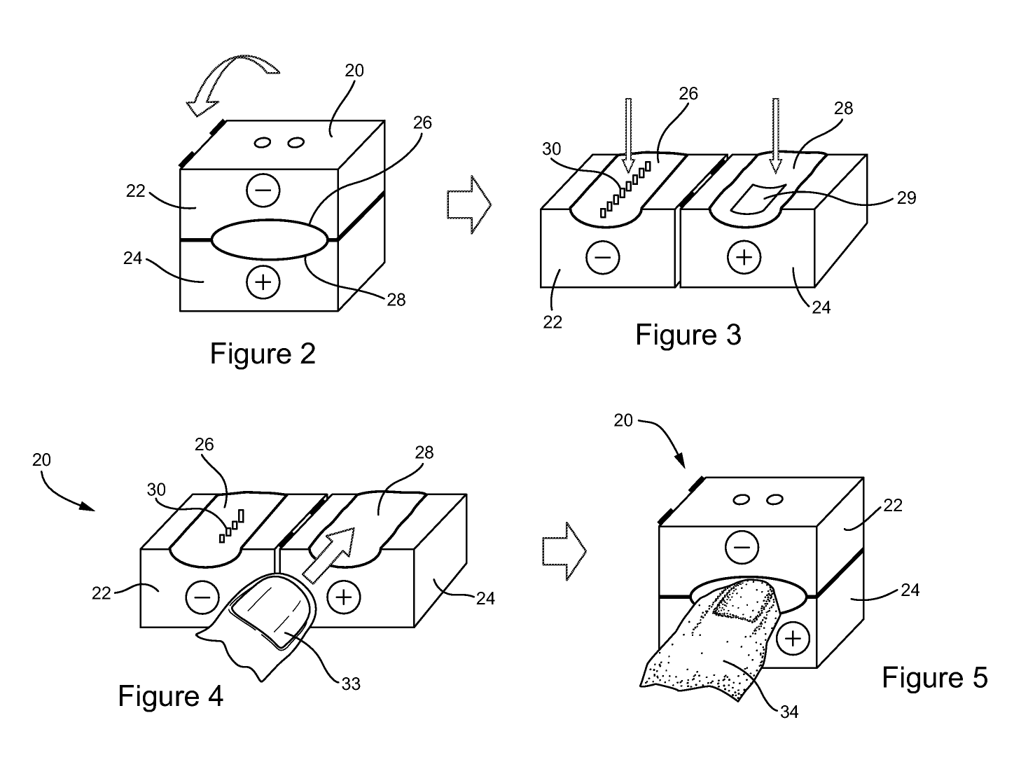

A technique has been developed to form a medicament matrix 14 that is individually sized to fit the toenail area to receive the onychomycosis treatment. This technique is disclosed in FIGS. 2 to 8 that show in sequence the operation an electrokinetic medicament applicator device 20 for applying medicament, e.g., onychomycosis medicament, to a toenail or finger nail. The device includes an upper housing 22 and a lower housing 24 that may be hinged together. The upper housing 22 includes a well 26 on a lower surface that faces the lower housing, when both housings are closed around a toe. The well 26 is arranged to receive the upper surface of the toenail and the toe. Similarly, a well 28 is formed on an upper surface of in the lower housing 28, wherein the well 28 faces the well 26 in the upper housing with the upper and lower housings are clamped together, as shown in FIG. 2.

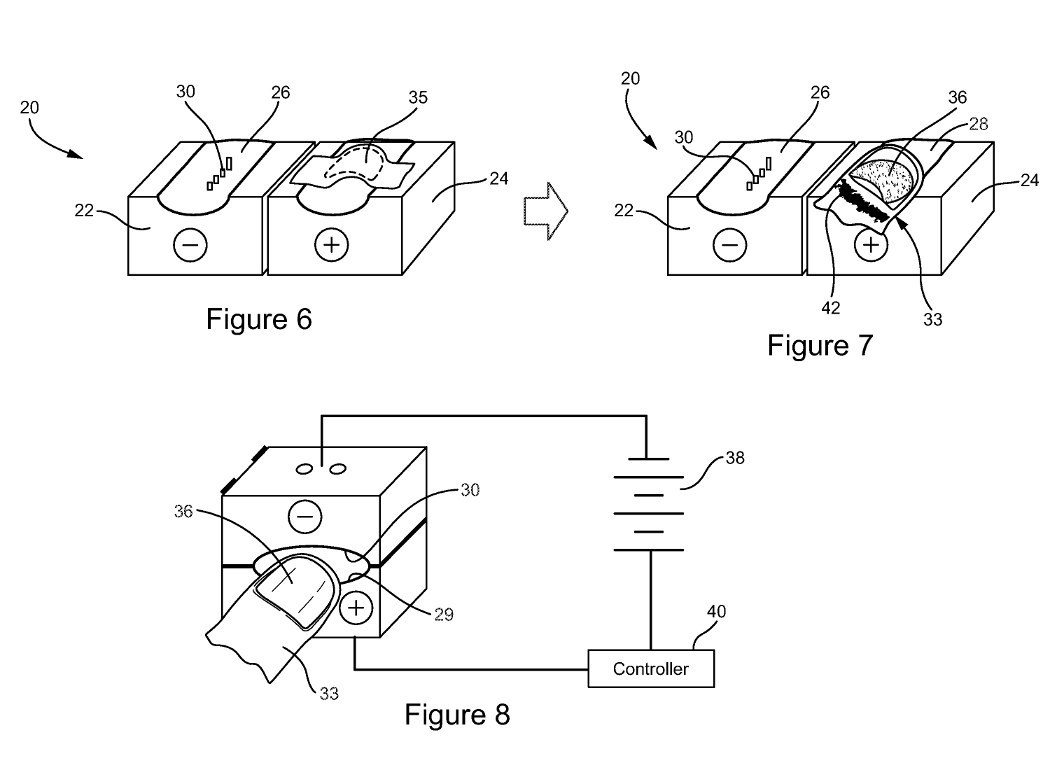

The wells 26, 28 include conductive surfaces and/or features called electrodes. The electrode(s) 30, e.g., negative electrodes, in the well 26 of the upper housing are to be adjacent one side of a medicament matrix to be applied to the toenail area. The electrode 29, e.g., a positive electrode, in the well 28 in the lower housing is intended to contact the underside of the toe. The surface of the well 28 of the lower housing 24 may form the positive electrode. It suffices to say that the polarity of these electrodes may be reversed to accommodate the charge on the active pharmaceutical agent to be delivered iontophoretically. When the toe is in the housings, the skin pad of the toe is in contact with the electrode 29 on the surface of the well 28 to provide an electrical connection with the skin on the underside of the toe. Similarly, the upper well 26 includes electrodes 30, e.g., deformable conductive stubs, that contact a medicament matrix 35 (see FIGS. 6 and 7) seated in the well 26 of the upper housing. The electrodes 30 may be individually controlled or have current limiters to ensure that current in any one of the electrodes does not exceed a maximum current. When current is applied to the electrodes 28, 30 current flows through the toe and medicament from the matrix is electrokinetically delivered into the nail area.

FIG. 4 illustrates the first step of inserting a toe 33 in the open well 28 of the lower housing 24. FIG. 5 shows the well 26 of the upper housing 22 closing over the toe and the well 28 of the lower housing 24. A polymer cast material is pumped into at least the well 26 of the upper housing and applied to the toenail. A polymer cast 34 is formed of at least the toenail and upper surface of the toe.

The cast 34 is formed by conventional casting techniques procedures such as used for fitting orthodontics or dental crowns and restoratives. The material for the casting 34, e.g., a sheet, powder or liquid, is placed in the well 26 of the upper housing 22 and over the toe. The cast material may be an electrically conductive medical grade polymer, e.g., silicon rubber with conductive additives. The cast material coats the interior surfaces of the upper well 26 and the upper surface of the toenail. The cast material solidifies or deforms to form a reverse impression of the toenail and the upper surface of the well 26, as is shown in FIG. 5.

The polymer cast 34 of the toenail and a portion of the upper surface of the toe is accessible by separating the upper and lower housings. A sheet of a medicament matrix 35 carrying a medicament, such as onychomycosis medicament, and compositions to facilitate the electrical carrying of the medicament is trimmed to conform to the toenail area as indicated by casting.

As an alternative to forming the casting 34, the matrix sheet 35 may be applied directly to the actual toenail as shown in FIG. 6. Specifically, the untrimmed matrix sheet may be applied directly to the toe nail and the user traces an outline of the nail on the sheet. The sheet is removed from the nail, and the user trims the sheet by cutting along the outline on the sheet. The trimmed matrix sheet 36 is reapplied to the toenail area. In this alternative process to form a trimmed sheet, it is not necessary to form a casting of the toenail.

As shown in FIG. 7, the trimmed matrix sheet 36 forms a medicament matrix that covers only the toenail. The soft skin tissue around the toe nail is not covered by the trimmed matrix sheet 36. Using the trimmed matrix sheet, lower impedance peripheral soft tissue is avoided during application of current and the risk of burns to the soft tissue is diminished. Alternately, a larger sheet may be applied to nail and soft tissue as long as current to the soft tissue is controlled to prevent excessive current density from harming the soft tissue.

The trimmed matrix sheet 36 is applied to the toenail and the toe is thereafter seated in the well 28 of the lower housing 24. As the well 26 of the upper housing 22 is closed over the toe, the electrode stubs 30 in the well of the upper housing 22 electrically contact the trimmed matrix sheet 36. The stubs press the trimmed matrix against the toenail. The reverse toenail casting ensures that the trimmed matrix 36 closely conforms to the upper surface of the toenail and makes good electrical contact with the entire surface area of the toenail. The casting material may be deformable and resilient so that as the upper housing 22 closes over the toe, the casting applies a force against the trimmed medicament matrix to conform the matrix to the toenail area.

In addition, the soft skin tissue adjacent the toenail may be painted with a non-conductive paint 42 prior to closing the upper housing 22 over the toe 32. The paint may be a conventional two-part medical-grade room temperature vulcanization (RTV) non-conducting silicone liquid that is applied to the soft skin tissue at the periphery of the toenail. RTV silicon is a conventional material and commonly used for medical purposes, such as for preparing dental casts. The RTV silicon may be packaged with a disposable hand-held brush-on device for application to soft skin tissue of the toe. The RTV silicon is painted on as a liquid and cures to for a protective film over the soft skin tissue in about five minutes, which is appropriate for out-patient service. The RTV silicon film prevents direct contact between a medicament matrix and the soft skin tissue adjacent a toenail to which the matrix is applied. The RTV silicon film also helps prevent excessive current flow to the soft skin tissue. The RTV silicon film 43 may be peeled off the toe and disposed of after electrokinetic application of the medicament to the toenail is completed.

As an alternative to painting RTV silicon, a thin, non-conducting membrane with medical adhesive backing would be applied to the toe. This may include medical grade tapes with adhesive already applied or may be formed of a material similar to that used during dental procedures to isolate a tooth. The adhesive backing is applied to the toe to secure the membrane to the toe. The portion of the membrane over the toenail is removed by tearing or cutting. The nail is exposed and the soft skin tissue around the nail is covered by the non-conductive membrane. The trimmed medicament matrix sheet 36 is applied to the toenail and possibly partially over the RTV silicon film or non-conductive membrane 42, which protects the soft skin.

FIG. 8, the electrical current is applied to the positive and negative electrodes in the wells and current flows through the medicament matrix 36 (formerly casting). The current electrokinetically delivers the medicament from the matrix into the nail to treat the infection. Current is supplied by a power source 38, such as a portable battery, and the application of the current is controlled and monitored by a computer controller 40.

The power source and controller may be housed in a portable housing having a user interface, e.g., actuation buttons and a display that allows a user to activate the delivery of medicament and to read results of the delivery such as successful application of current. The controller 40 may comprise the power system 38, such as batteries, a microcontroller for monitoring certain conditions, such as whether a excessive current is applied to the medicament matrix, and controlling the application of current to the active electrode, and conductive circuits connecting the power supply, microcontroller, actuator switch, and electrodes in the upper and lower housings. The controller, when actuated, applies current to each of the plurality of electrode segments of the active electrode. The current applied to the electrode segments may be, for example, on the order of 660 microAmps (uA). A current path includes the power source 38, the positive and negative electrodes 29, 30, medicament matrix 36 and toe and toenail of the patient, e.g., a mammalian user, and the controller 40.

An advantage of the sheet trimming technique and/or casting technique is that a medicament matrix is tailored to the topology of a toenail. Trimming a medicament sheet allows the shape of the trimmed medicament matrix to be specific to the toenail area to which the matrix is to be applied. Casting provides a means for forming a reverse impression of the toenail area that can be used to press a sheet of medicament matrix against the toenail during electrokinetic delivery of medicament or be used directly as the medicament matrix. Because the casting is an accurate reverse impression of the toenail area, the medicament matrix conforms to the toenail area and is in contact with the entire upper surface of the toenail.

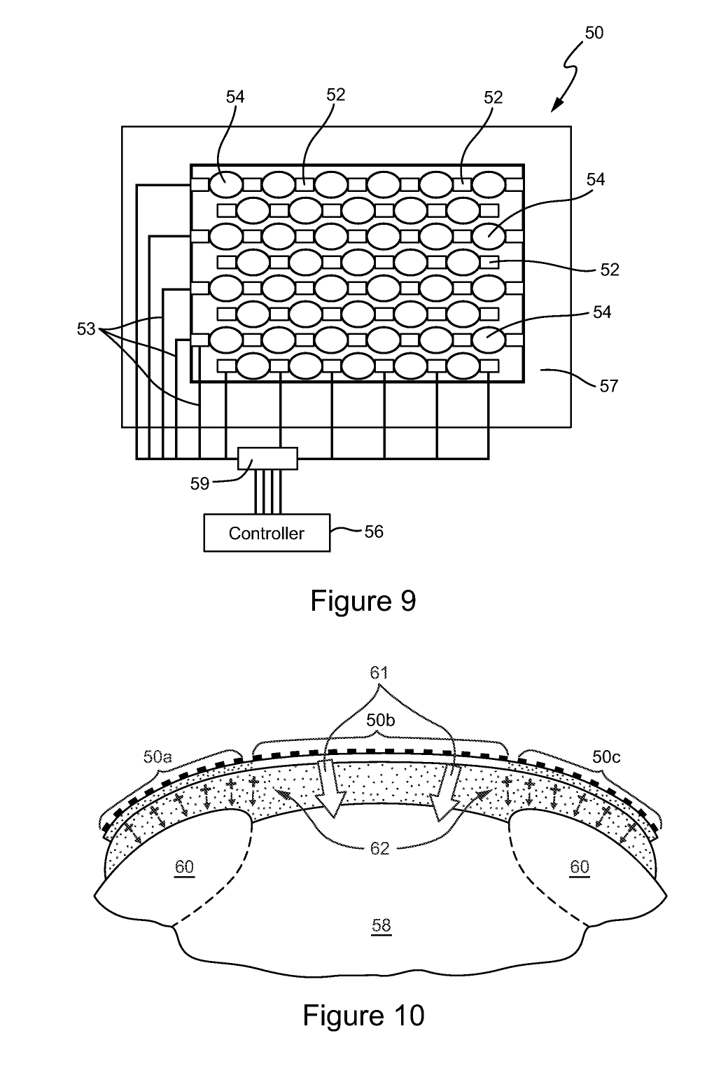

FIG. 9 is a schematic diagram of a flexible and perforated electrode grid 50 having a two-dimensional array of electrodes 52 and perforations, e.g., apertures 54. The electrodes grid 50 is a deformable sheet that conforms in x, y and z directions to the surface of the toenail and upper surface of the toe. The electrodes 52 are electrically coupled to a controller 56 that can individually control each electrode. The controller senses the current through each electrode and can adjust the voltage or current applied to each electrode. The perforations 54 in the grid allow medicament from a medicament matrix to flow through the grid and to a toenail.

FIG. 10 is a schematic diagram of an electrode grid wherein the electrodes are for purposes of illustration grouped as electrodes 50a, 50b and 50c. The electrode grid is applied to a toenail 58 and to the soft skin tissue 60 adjacent the toenail. Current is applied to the electrodes 52 (FIG. 9) of the grid. Initially, a uniform voltage may be applied to all electrodes. A controller 56 senses the current at each electrode. A high current at an individual electrode indicates that the electrode is in electrical communication with a low impedance skin area, such as soft skin tissue or a cut in the skin. With respect to the electrodes 50a, 50c associated with the low impedance skin area (as indicated by the “+” symbols), the controller may limit the current through those electrode 50a, 50c to reduce the risk of excessive current being applied to the skin. The low impedance sites of the soft tissue and/or breaks in the skin are shielded electronically by the reduction in current to the electrode associated with the low impedance sites. The electrodes 50b over a high impedance skin area, e.g., a toenail, may continue to receive relatively high current levels, as determined by the controller.

The electrodes 52 are electrically coupled to the controller 56 through wires that extend from the electrodes to a border region 57 of the grid and to an electrical interface coupling 59 between the grid 50 and the controller 56. The border may be a rectangular flexible sheet having a generally open center region in which are the electrodes 52 and perforations 54 through the grid. Electrical wires 53 extend from the electrodes, through the border region 57 (See FIG. 9) and to the coupling 59. The wires are arranged so that each of the current through each electrode may be powered, sensed and controlled by the controller 56.

The controller 56 dynamically senses and adjusts current to each of the electrodes in the grid 50. The controller 56 may comprise a power system, such as batteries; a microcontroller for monitoring certain conditions, such as whether excessive current is applied to any of the electrodes 52, and controlling the application of voltage and/or current to the electrodes, and conductive circuits connecting the power supply, microcontroller, actuator switch, active electrodes, e.g., electrodes 52, and a counter electrode. The controller, when actuated, applies current to the electrodes 52 in the grid. The current applied to the electrodes may be, for example, on the order of 660 microAmps (uA). A current path includes the controller with power source, the active electrodes 52 in the grid, the medicament matrix 62, the toe and toenail 58 of the patient, e.g., a mammalian user and a counter electrode that may be attached, for example to the skin pad on the bottom of the toe with the toenail being treated with the medicament matrix 62.

The controller and grid effectively map the impedance of the toenail and skin below the grid. Using the impedance map, the controller ensures that current applied to those electrodes associated with high impedance areas (which are presumably associated with the toenail) are at a current level sufficient to electrokinetically deliver (see arrows 61) medicament from a medicament matrix 62 to the toenail.

The electrode grid 50 may be a reusable while the medicament matrix 62 may be disposable. Alternatively, the flexible grid could be placed within the drug patch, sensing current through the drug and adjusting the level of drug delivery via the array of electrodes 52.

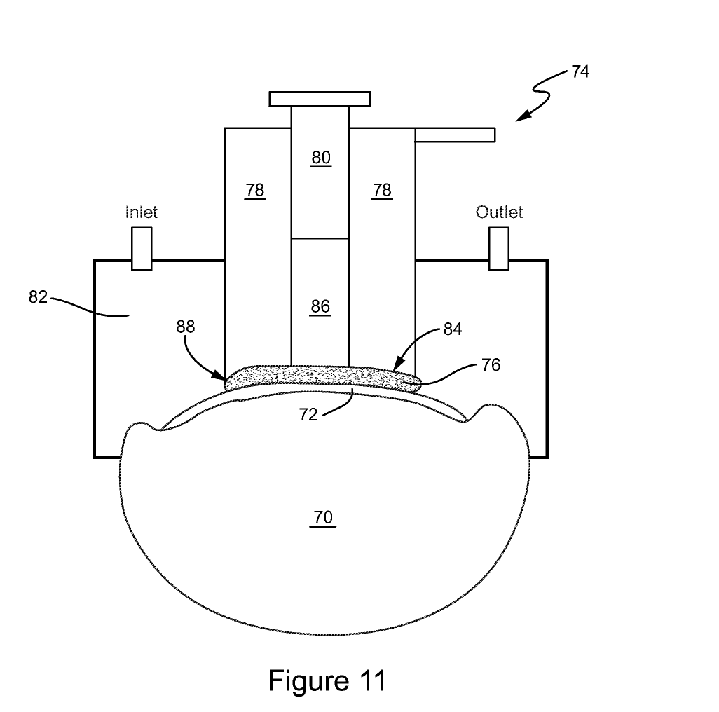

FIG. 11 is a front view of a toe 70 having a toe nail 72 and an electrokinetic medicament applicator 74 that includes a medicament matrix 76 applied to the toe nail, a cylindrical conductive positioner 78, a drug luer 80 inserted into an open end of the hollow positioner, and a compartment 82 for receiving room temperature vulcanizing (RTV) rubber that shields the soft skin tissue of the toe from excessive electrical current applied by the positioner.

The annular front face 84 of the conductive positioner 78 is applied to an upper surface of the drug matrix that is opposite to a lower matrix surface applied to the toe nail. The annular front face 84 has a slightly smaller surface area than the surface area of the toe nail, e.g., less than 100% the area of the toenail but preferably greater than 75% the area of the toenail. Further, the front face 84 may be contoured or otherwise shaped to conform to the generally curved surface of a toe nail.

The drug matrix 76 may be a nonwoven porous material, foam or other retentive matrix for at least temporally storing the medicament or providing a passage of the medicament from the interior passage 86 of the positioner that is open at the front face 84 to the medicament matrix 76. Alternately, the matrix may be a previously applied polymer film, viscosified drug formulation, or phase-change polymer described in this patent. The matrix may be formed with an array of active electrodes, such as is shown in FIG. 9. The array of electrodes may be electrically connected at the periphery of the array to the front face of the positioner, which may be a conductive cylinder that provides an electrical connection between the active electrode and a positive terminal of an electrical power source.

The matrix is positioned at or near the center of the toenail and provides a base against which to position the front face 84 of the positioner 78. An annulus, such as a small “O”-ring, 88 may extend around the perimeter of the matrix and provide a brace between the toenail and front face 84 preventing excessive compression of the matrix by the positioner.

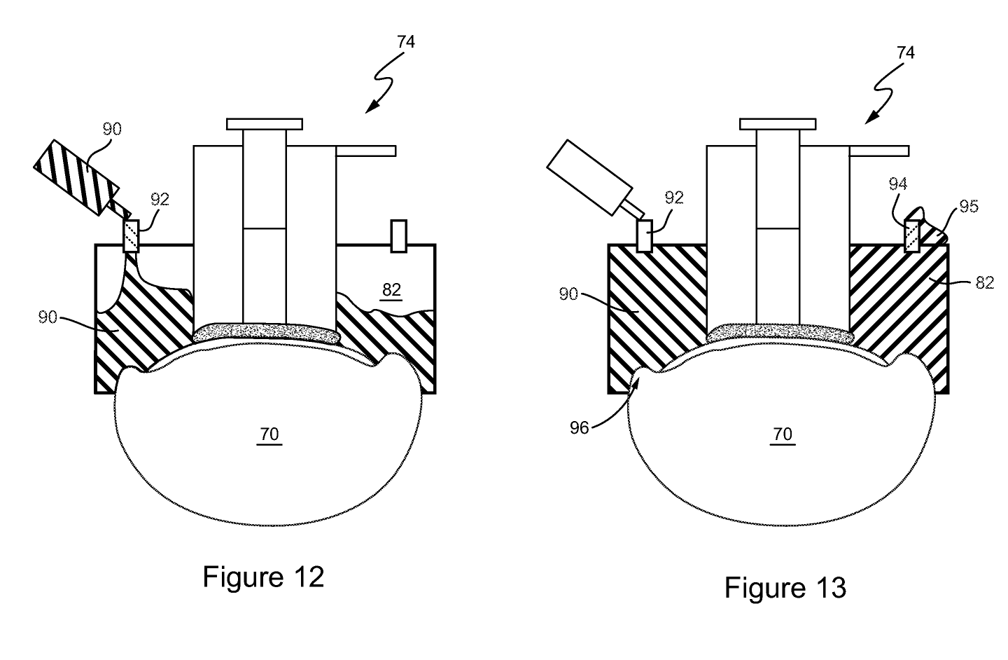

FIG. 12 is a front view of a toe 70 with an electrokinetic medicament applicator 74 where the compartment 82 is being filled with RTV rubber 90. The rubber is supplied through an upper inlet 92. When the compartment 82 is filled with liquefied rubber, a small amount of the rubber is discharged from the outlet 94 of the housing.

FIG. 13 shows the discharge 95 of rubber which indicates to the user that the compartment is filled. When the compartment is filled, the RTV rubber provides an insulating shield protecting the soft skin tissue 96 adjacent the toenail from excessive current from the conductive positioner.

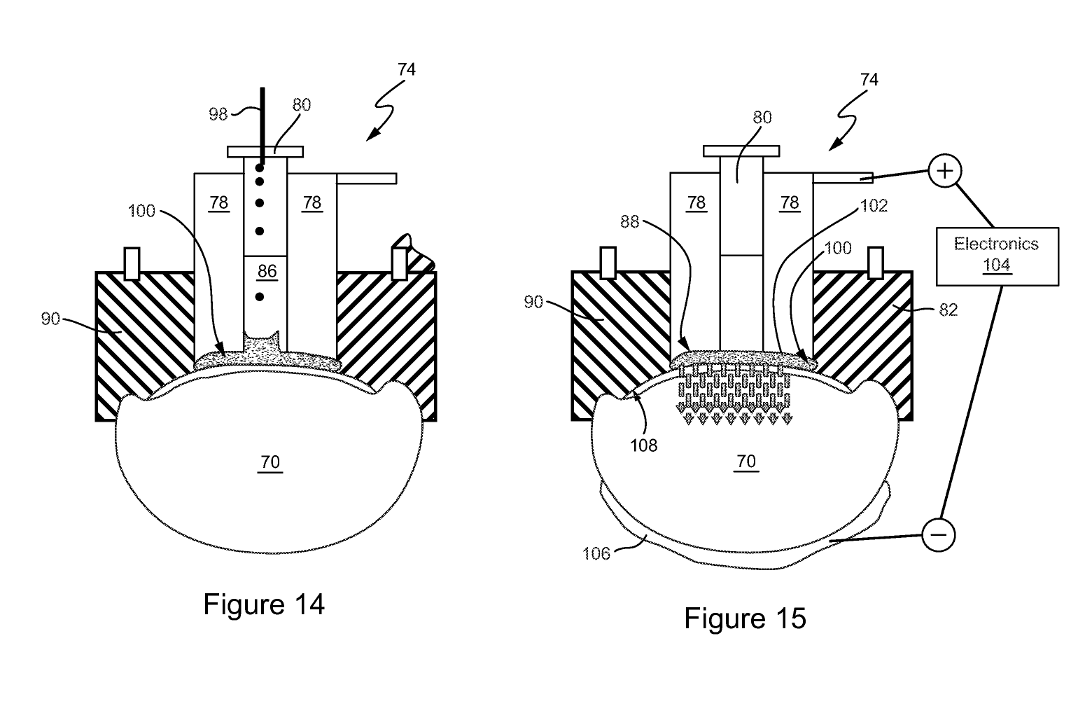

FIG. 14 is a front view of a toe 70 with a toenail having the applicator 74 applied to a medicament matrix 100 that is being saturated with medicament passing through the passage 86 of the positioner 78. The medicament is injected through the luer connector 80 that couples the passage 86 of the positioner to a drug (medicament) supply line 98. As medicament flows into the passage 86 and flows to the medicament matrix 100, the porous matrix temporarily stores the medicament adjacent the toenail. Once the matrix is receives the medicament, the drug line 98 may be detached from the luer connector 80.

FIG. 15 is a front view of the toe 70 with the applicator 74 applied to the toenail. The applicator is ready to deliver the medicament electrokinetically into the toenail to treat, for example a fungal infection of the nail. The soft skin tissue is protected by the RTV rubber 90 in the container box 82. The medicament matrix 100 is soaked or otherwise loaded with medicament. The positioner 78 is connected to a positive electrode 102 and the underside of the toe 70 is connected to a negative electrode 106, although the polarity of the electrodes may be switched. The positive and negative electrodes are connected to electronics that include a power source, e.g., a battery and control electronics, e.g., a timer, current limiter and a manually operated switch.

When sufficient current is applied to the conductive positioner, the front face 88 of the positioner applies current to the medicament pad. Current flows through the pad, the toe and to the negative electrode 106 and then to the electronics 104 that supplied current to the positive electrode 102. The flow of current through the medicament matrix and into the toe nail electrokinetically delivers the medicament from the matrix into the nail. After an appropriate time for application of the current, the electronics automatically turns off the current and the treatment to the nail is completed.

During application of the current, the insulating RTV rubber 90 prevents current flow from the positioner front face 88 or the matrix 100 to the soft skin tissue 96 adjacent the toe nail. The RTV rubber solidifies and preferably does not adhere to the toenail or skin of the toe. After the first treatment is completed, the applicator 74 may be removed. The front surface 108 of the RTV rubber is effectively a reverse molded shape of the upper surface of the toe and toenail. The applicator with the solidified rubber may be reused on the toe during subsequent electrokinetic treatments. The molded rubber provides an insulating shield for the skin of the toe, and assists in directing the medicament to the toe nail.

In a further embodiment, an iontophoretic applicator delivery device delivers terbinafine to a nail. The applicator device includes port to receive a solution including terbinafine and a nonionic pluronic polymer. The polymer with the terbinafine is applied by the device as a sol, e.g., colloidal suspension, to a nail of the patient. The pluronic polymer with terbinafine flows over and coats the nail. After coating the nail, the polymer undergoes a phase transition in which the sol transforms to a gel coating the nail. The gel forms a conductive coating on the nail that is infused with the medicament, e.g., terbinafine. An electrode applies an electrical current to the gel to iontophoretically deliver medicament, e.g., terbinafine, to the nail.

The applicator device preferably: (i) has a low profile and is adaptable to the various toe and nail sizes that may be found on human patients; (ii) is easily applied to a nail by a practitioner, e.g., a health care provider; (iii) is disposable after one use of applying the medicament to the nail; (iv) is inexpensive to produce; (v) includes an electrode integral with the applicator device; (vi) includes a wireless data connection to a computer or other data collection device, and (vii) includes a stay-in-place drug formulation that may be included with the device or is easily mounted on the device. The applicator device described herein may be embodied to include all, or just one or more, of these preferences for the device.

The applicator device preferably includes an applicator container for delivering to a nail nonionic polymers and mixtures of polymers noted for their phase change characteristics. A preferred nonionic polymer and polymers mixture includes pluronic polymers comprising polyoxypropylene (POP) and polyoxyethylene (POE) blocks demonstrating lower critical solution temperature (LCST) phase transformations when formulated appropriately.

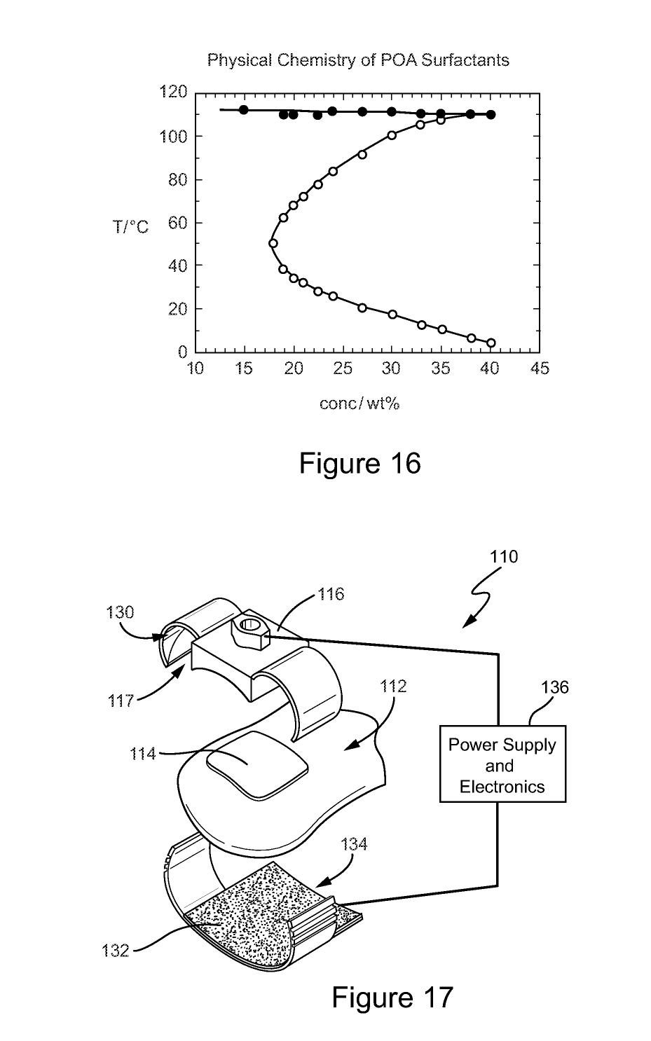

FIG. 16 shows a typical phase diagram of an E₉₉P₆₉E₉₉ block copolymer pluronic, referred to as F-127. This copolymer pluronic has been conventionally used by tissue engineers in developing surgical products. At 20% by weight in water, this polymer undergoes a sol (colloidal particles) to gel phase change at or near body temperature that immobilizes the polymer.

The phase change from sol to gel by the pluronic polymer material is desirable for purposes of forming a casting on the surface of a nail that serves as the applicator of the current and the medicament to the nail. The pluronic polymer material may remain in a sol phase prior to application to the nail. When the pluronic polymer is applied to the nail, the polymer is in the sol phase and flows over the surface of the nail. After forming a coating over the nail, the pluronic polymer changes phase and forms a gel that conforms to the nail. The gel formed by the pluronic polymer is relatively immobile. The phase change from sol to gel and the immobilization of the gel are desirable because the characteristics provide for the polymer to wet and spread over the nail surface and then form a leak proof gel after the polymer as undergone a phase change, e.g., “set”.

Terbinafine and attendant excipients (collectively the medicament) are infused into the nonionic pluronic polymer and loaded into a medicament delivery device. The polymer with medicament is held in a sol (colloidal solution) phase in the device until the application of the device to a toenail. The applicator applies the polymer with medicament as a liquid (in a sol phase) to the nail. At a certain temperature, such as in a range of 32 degrees Celsius (C) to 37 degrees C., the liquid polymer gels on the nail and provides a conductive matrix for delivery of the active medicament. After a requisite coulombic dose of terbinafine has been delivered to the nail through the gelled polymer applied to the nail, the therapy is ended (such as by ceasing the application of electrical current) and the gel-filled applicator is removed and discarded.

Other classes of phase-change polymers can be used as well for the application of a medicament to a nail or other treatment site on the body of a patient. For example, isopropylacrylamide-coacrylamide, poly(n-vinylcaprolactam), and hydroxypropylmethacrylate copolymers exhibit a lower critical solution temperature (LCST) and behave like the pluronic polymer described above. Other polyelectrolytes are responsive to electric fields that can be used to trigger release of ionically associated actives from the polymer network. The terbinafine cation, therefore, can be bound by a polyanionic matrix and released to the medium through application of electric current during iontophoresis.

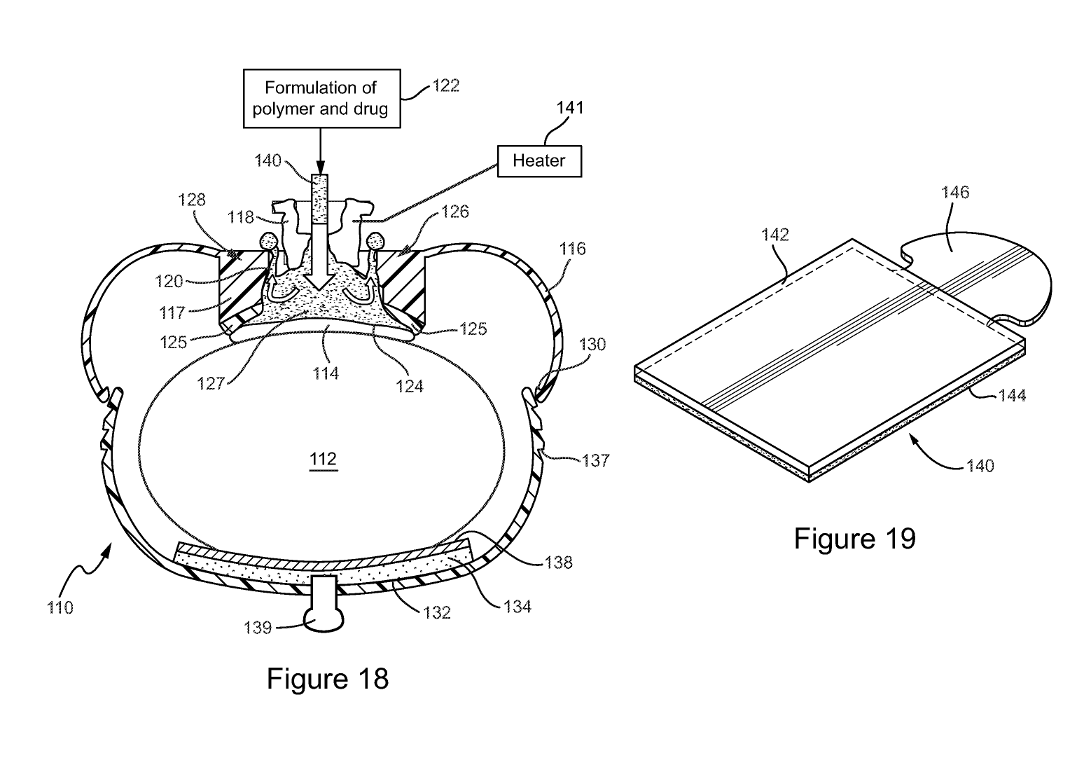

FIGS. 17 and 18 show an example of an applicator device 110 for applying a phase changing polymer with medicament to the nail of a patient. FIG. 17 shows an exploded view of the applicator device and a toe 112 with a nail 114 to which the applicator device is to be applied. FIG. 18 shows in cross-section a front view of the applicator device 110 applied to the toe and toenail. The applicator device 110 is designed to simplify preparation of onychomycotic nails for terbinafine treatment.

The upper portion 116 of a housing 117 of the applicator device includes a fill port 118 and vent 120. The formulation 122 of polymer and medicament is added to a container 124 in the housing 117 through the port 118 until excess formulation is discharged out of the container through the vent 120. The fill port may be in the form of a luer connection that is generally cylindrical and includes an upper inlet to receive a connection from the container, e.g., a syringe with an outlet port connectable to the luer connection.

The applicator housing 117 may be rectangular in shape and having an interior container 124, e.g., chamber, that has an open bottom facing the toenail. The vent 120 and port 118 are preferably mounted on an upper portion 116 of the applicator housing. The vent allows excess formulation injected into the container 124 to flow up and out of the applicator through the vent.

The luer fill port 118 may also serve as the anode and power connector for the electrical portion of the applicator housing and particularly to provide an electrical connection to the formulation injected into the container. The luer may be conductive, e.g., metallic, and connectable to an electrical power source and control electronics assembly 136. The electrical current from the power source is applied via the luer fill port to the formulation injected into the applicator container 124. In this manner, the luer fill port serves as an anode which applies electrical current to the formulation in the applicator container 124.

The applicator housing may include a mask 125 that forms the sidewalls of the housing. The mask may be a deformable plastic material and shaped as a rectangular ring. The size of the mask may be selected or trimmed such that the lower surface of the mask conforms to the upper surface of the toe. The mask deforms to conform to the toe and toenail surface when pressed against the toe. The mask has an open interior space to allow the formulation to flow down on and conform to the toenail. An interior rim 127 of the mask extends around the open interior space. The rim 127 overlies the toenail and generally extends around the perimeter of the toenail. The rim 127 confines the formulation to the toenail and away from the soft skin tissue adjacent to the toenail.

The mask may be held in place by an upper chamber housing 128. The interior of the upper chamber housing forms the container 124 for the formulation. The housing 128 may be a soft, closed-cell foam panel attached to a lower surface of the upper portion 126 of the applicator 110. The upper chamber housing is pressed by the upper portion 126 to the mask 125 and toe. The rim of the mask 125 and the container 124 define a chamber to receive the formulation and apply the formulation to the toenail.

The applicator device 110 may include an upper and lower bracket sections 130, 132 that fit around the toe, and are joined and ratcheted to a snug but comfortable fit to the toe. The lower bracket section 132 contains a conductive pad 134 that provides an electrical connection between the lower pad of the toe and the electrical power supply and control electronics 136 that are also connected the luer connector that serves as an anode. The control electronics may include a processor programmed to apply current from the power supply to the anode (luer port 118) and cathode (pad 134) of the applicator device. The conductive lower pad 134 may form a cathode connector for the applicator device. A conductive gel 138 may be used to improve the electrical connection between the bottom of the toe and the cathodic conductive pad 134. An electrical contact 139 may provide a connection between the pad 134 and gel 138 with the power supply and control electronics 136.

In use, the mask 125 is selected or trimmed to fit the toenail. The mask is applied to bottom of the upper chamber housing 128 or is applied directly to the toe such that the rim 127 of the mask extends around the perimeter of the toenail. The applicator device 110 is applied to the selected toe by connecting the upper and lower brackets 30, 32 together. The brackets may be ratcheted 137 together to press the upper chamber housing and mask tightly against the toe.

The formulation 122 of Terbinafine, attendant excipients (collectively the medicament) and a nonionic pluronic polymer may be cooled to a prescribed temperature and infused into the applicator container through the luer fill port. The formulation 122 flows through a conduit, e.g., a tube, 140 that is connected to the luer fill port 118. The formulation fills the applicator container 124 and covers the toenail. The filling of the container 124 is ceased when formulation begins to flow out the vent 120.

The formulation behaves as a solution at a temperature below about 37 degrees Celsius (C) (and most preferably below about 25 degrees C.), provided an appropriate concentration of water is in the formulation. Accordingly, the formulation is preferably maintained below 35 degrees C. or 25 degrees C. until it is injected into the applicator container 124. After being infused into the applicator container and coating the toenail, the formulation warms to above 35 degrees C. or preferably above 25 degrees C. and thereby forms a gel. The formulation may be warmed as it flows into the container or by a heater 141 applied to the upper chamber housing 128. When heated, the formulation forms a conductive solid gel applicator conforming to the toenail.

The applicator device 110 is activated by the control electronics that applies current to the anode and cathode 118, 134 and the resulting electrical current delivers the terbinafine to the toenail. In one embodiment, the control electronics 136 may be configured to apply a current to the anode and cathode electrodes 118, 134 for a period of hours, such as during an overnight period during which the applicator deliveries terbinafine to the toenail. For an applicator device delivering terbinafine during a period of hours, such as overnight, the current applied to the electrodes 118, 134 may be in a range of 0.025 milliamperes (mA) to 0.05 mA for 6 to 8 hours. Such a current level and period of time should be sufficient to deliver terbinafine to a toenail at voltages within the capability of onboard electronics.

The housing 117 may be modified to accommodate more than one combination of upper chamber housing and mask. Multiple combinations of upper chamber housings and masks may be applied to two or more toes simultaneously. The electronics and power supply 126 may be multi-channel system such that each applicator for each toe may be separately activated by the application of current to selected ones of the toes.

FIG. 19 schematically shows an applicator pad 140 the viscous gel formulation 142 and an electrode panel 144 backing the gel formulation. The applicator pad 140 may be applied to a wide area treatment site, such as a wide area of the skin or to a toenail or other high impedance treatment site. The electrode panel may be electrically connected to a power supply and control electronics. The electrode panel may be entirely flat, curved to conform to a toenail and/or have a rim around its perimeter, where the rim forms a sidewall to the gel formulation applied to the panel. The electrode panel may be flexible and deformable so that it may conform to the treatment site, such as a toenail or skin surface.

Casting of the formulation directly on the electrode panel may be performed by conventional and well known casting techniques. The viscous gel formulation is cast directly onto the electrode panel, such as by heating the formulation to a liquid phase and pouring the liquid formulation on the panel. To cast the formulation, the formulation may be poured directly on the panel. The panel may be placed in a bracket or other casting device to ensure that the formulation as cast is aligned with an on the electrode panel. The panel may be cooled to, for example, ambient temperature, such as below 35 degrees Celsius, to solidify the gel formulation to a flexible layer on the electrode panel.

The formulation may be cast directly on the electrode panel in an automated casting process for producing applicator pads. During the casting process, the formulation cast on each of the electrode panels may contain a carefully controlled amount of a particular medicament. The casting process may produce applicator pads having gel formulations with various medicament dosages and various medicaments. The applicator pads may each be packaged with labels indicating the medicament and medicament dosage in the gel formulation on the pad.

When the applicator pad is to be applied to a treatment site, the packaging for the pad is removed and the pad is connected to a electrokinetic device having a power supply and control electronics, which is in turn connected to a counter electrode applied to the user. The electrode pad may have a tab 146 which is coplanar with the panel 144 and connects to a connector of the power supply and control electrodes. Further, the electrode panel and gel formulation may be trimmed to conform to the treatment site, such as a toenail. For example, shears or scissors may be used to trim the applicator pad. The pad may be applied to the skin by an adhesive surface on the front of the cast gel formulation or by an adhesive strip applied over the back of the applicator pad after the pad is positioned on the skin.

The electrode panel 144 may be formed of conventional electrode material used for application to the skin, such as materials used to form electrodes for EEG and EKG electrodes. The electrode panel may be entirely conductive or a laminate formed of a network of conductive wires and a flexible substrate. Materials for forming the electrode panel may be conductive polymers and other electrode materials, such as silver or silver-chloride. These materials are conventionally formed into electrodes by roll processes that may be easily adapted to cast a gel formulation on the electrodes. The electrode panel is conductive entirely or at least along on a surface which is in contact with the gel formulation.

The electrode panel provides electrical current to the backside of the gel formulation cast on the panel. The current from the panel flows through the gel formulation and into the skin and treatment site. The current causes charged substances in the gel formulation, such as the medicament or carrier particles for the medicament, to be iontophoretically delivered to the treatment site in the skin or toenail. The current flows from the electrode panel, gel formulation and treatment site and through the body to a counter electrode that is in contact with the body of the user and with the same power supply and control electronics as is the electrode panel.

The gel formulation preferably forms a solid medicament layer applied to the electrode panel. This medicament layer preferably is flexible to conform to the skin or a toenail and has an adhesive quality that causes the layer to adhere to the skin or toenail while medicament is electrokinetically delivered to the treatment site. The gel formulation is preferably a composition that has a high degree of stability and is compatible with the electrode panel. Further, the gel formulation is a carrier matrix for a medicament, such as an antifungal compound. The preferable gel formulation suitable for iontophoresis may include: Terbinafine hydrochloride (0.1 to 10.0% w/w (percent by weight)), 95% alcohol (5.0 to 30.0% w/w), anti-oxidant (0.1 to 1.0% w/w), chelating agent (0.1 to 1.0% w/w), solubilizer, such as polysorbate 80 (1.0 to 5.0% w/w), co-solvent, such as glycerin (10 to 40% w/w), a thickening agent, cellulose based such as hydroxyethylcellulose, xantham gum (0.1 to 1.0% w/w), film forming block polymer agent, such as polyethylene oxide (1.0 to 10% w/w), a temperature sensitive hydrogel former, such as poloxamer (5 to 30% w/w), a permeation enhancer (0.1 to 2.0% w/w), a preservative, and the remaining amount being purified water.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

We claim:

1. A method to electrokinetically deliver a medicament to a nail of a mammalian user, the method comprising:
forming a cast of the nail of the user wherein the cast is a reverse impression of the nail area;
applying a medicament matrix to the nail area;
sandwiching the medicament matrix between the cast and the nail area;
pressing the medicament matrix to the nail area using the cast;
applying electrical current to an electrical current path extending through the medicament matrix and the nail area, and
delivering medicament from the matrix into the nail area by electrokinetic transporting the medicament along the current path.

2. The method in claim 1 wherein the nail is a toenail.

3. The method in claim 1 further comprising extending an electrode through the cast and to the medicament matrix, wherein the electrical current path includes the electrode.

4. The method in claim 2 further comprising a second electrode applied to an underside of the toe and the current path includes the second electrode and the underside of the toe.

5. The method in claim 1 further comprising forming the cast in an assembly having an upper housing with a well and a lower housing with a well opposing the well in the upper housing, wherein cast is formed by applying casting material to at least the well of the upper housing and closing the upper housing over the lower housing while the nail is sandwiched between the wells.

6. The method in claim 1 wherein applying the medicament matrix further comprises trimming the medicament matrix to conform to the nail area.

7. The method in claim 1 further comprising applying a non-conductive layer to soft skin tissue adjacent the nail prior to the application of the electrical current.

8. The method of claim 7 wherein applying the non-conductive layer includes painting a liquid non-conductive silicone liquid to the skin tissue and curing the liquid to form a film on the skin.

9. The method of claim 7 wherein applying the non-conductive layer includes applying an adhesive backed non-conductive sheet to an upper surface of a toe and removing a portion of the sheet over the nail and retaining a portion of the sheet over the skin.

10. An electrode grid and medicament matrix assembly comprising:
the electrode grid including flexible array of electrodes and perforations through the grid and array of electrodes;
a controller sensing current at each of the electrodes and adjusting the current to each electrode, and
the medicament matrix adjacent the electrode grid.

11. The electrode grid and medicament matrix assembly of claim 10 wherein the medicament matrix is applied to a toenail and is sandwiched between the electrode grid and toenail.

12. The electrode grid and medicament matrix assembly of claim 10 wherein the electrode grid is a flexible sheet.

13. The electrode grid and medicament matrix assembly of claim 10 wherein the electrodes are arranged in a two-dimensional array.

14. The electrode grid and medicament matrix assembly of claim 10 wherein the perforations are interposed between the electrodes.

15. An iontophoretic drug delivery system for the treatment of nail infections comprising:
an applicator housing which directs and localizes a formulation of medicament and a phase transforming polymer to a nail of a patient, wherein the formulation behaves as a solution at a temperature below about 35 degrees C. and behaves as a gel above about 35 degrees C., and wherein the formulation is applied as a solution to the nail and transformed to a gel after application to the nail and the system includes integrated electrical connections and electronics for the application of therapeutic current to deliver the medicament in the formulation to the nail.

16. An iontophoretic drug delivery system for the treatment of nail infections comprising:
an applicator housing which directs and localizes a formulation of medicament and a phase transforming polymer to a nail of a patient, wherein the formulation behaves as a solution at a temperature below about 25 degrees C. and behaves as a gel above about 25 degrees C., and wherein the formulation is applied as a solution to the nail and transformed to a gel after application to the nail and the system includes integrated electrical connections and electronics for the application of therapeutic current to deliver the medicament in the formulation to the nail.

17. An iontophoretic drug delivery system for the treatment of nail infections comprising:
an applicator housing releasably applied to the nail, wherein the housing includes a container or chamber having an opening adapted to face the nail and a port above the opening through which a formulation is injected into the container or chamber, and
the housing includes a electrical connection through which electrical current is directed from a power source to the formulation in the container or chamber,
wherein the formulation includes a medicament to be delivered iontophoretically to the nail and the formulation behaves as a solution at a temperature below about 35 degrees C. (and most preferably below about 25 degrees C.) and behaves as a gel above about 35 degrees C. (and most preferably above about 25 degrees C.), and
wherein the formulation is applied as a solution to the nail and transformed to a gel after application to the nail.

18. The iontophoretic drug delivery system of claim 17 further comprising a bracket attached to the applicator housing and wherein said bracket is releasably secured to a digit having the nail.

19. The iontophoretic drug delivery system of claim 18 wherein the digit is a toe of a human patient.

20. A method to deliver a medicament to treat infections on a nail of a patient comprising:
applying an applicator housing to the nail wherein an opening in a container or chamber of the housing abuts the nail;
injecting a formulation of the drug and a phase transforming polymer into the container or chamber of the applicator housing, wherein the formulation is in a solution phase while being injected;
the formulation in the solution phase covers the nail at least where the opening abuts the nail;
the formulation transforms from the solution phase to a gel after covering the nail;
applying an electrical current to the formulation in the gel phase, wherein the current flows into the nail, and
delivering the medicament to the nail due at least in part to an iontophoretic delivery of the medicament.

21. The method of claim 20 wherein the transformation of the formulation occurs due to a change in temperature of the formulation.

22. The method of claim 21 wherein the formulation behaves as a solution at a temperature above 50 degrees Celsius and behaves as a gel below about 50 degrees C.

23. The method of claim 21 wherein the formulation behaves as a solution at a temperature above 35 degrees C. and behaves as a gel below about 35 degrees C.

24. An applicator pad comprising:
an electrode panel, and
a medicament layer on the electrode panel, wherein the medicament layer is a gel formulation cast on the electrode panel.

25. The applicator pad as in claim 24 wherein the electrode panel is a flexible panel having a connector to provide electrical connection to a power source.

26. The applicator pad as in claim 24 wherein the gel formulation is a composition comprising: Terbinafine hydrochloride and a cellulose or gum based viscosity modifier, acrylic acid based tackifiers, and a temperature sensitive block polymers.

27. A method of forming an applicator pad comprising:
forming an electrode panel having a conductive surface electrically connected to a connector attached to the panel;
casting a gel formulation on the electrode panel, wherein the gel formulation is applied to the electrode panel is liquid form and solidifies on the electrode panel.

28. The method of claim 27 wherein the casting of the gel formulation includes heating or cooling the gel formulation to a liquid state and applied to the panel as the liquid, and cooling or heating the panel to solidify the gel formulation on the electrode panel.

29. The method of claim 28 wherein the gel formulation is cooled to a liquid state and heated to solidify the gel formulation on the electrode panel.
